(19) United States
(12) Patent Application Publication
(10) Pub. No.: US 2023/0306523 A1
(43) Pub. Date: Sep. 28, 2023

(54) METHOD FOR OBTAINING INSURANCE CLAIM MATERIALS, BUSINESS SERVER AND SYSTEM

(71) Applicant: BOE Technology Group Co., Ltd., Beijing (CN)

(72) Inventor: Yanli XIN, Beijing (CN)

(21) Appl. No.: 17/607,818

(22) PCT Filed: Dec. 21, 2020
(86) PCT No.: PCT/CN2020/137852

§ 371 Date: Oct. 29, 2021

(51) Int. Cl. G06Q 40/08; G16H 40/20

(52) U.S. Cl. CPC G06Q 40/08; G16H 40/20

(57) ABSTRACT

A method for obtaining insurance claim materials, a business server and a system. The method includes: receiving insurance product information purchased by a target patient returned by insurance server; comparing diagnosis and treatment information of the target patient to determine whether a comparison result meets a claim prohibition condition; determining a list of target claim materials based on insurance product information and a list of designated claim materials when comparison result does not meet the claim prohibition condition; obtaining, from a hospital server, target claim material information corresponding to the list of target claim materials, sending the target claim material information to a terminal device. The diagnosis and treatment information is compared with the insurance product information to determine whether the comparison result meets the claim prohibition condition. The target claim material information is sent to a terminal device when the comparison result does not meet the claim prohibition condition.

### TECHNICAL FIELD

The present disclosure relates to the field of computer technologies, and more particularly, to a method for obtaining insurance claim materials, a business server and a system.

### BACKGROUND

With the improvement of people's living standards, people's insurance awareness has also increased, and more and more people choose to purchase medical insurance for themselves or their families.

Generally, after patients come to hospitals for diagnosis and treatment, they pay the hospitals for medical costs and obtain claim materials from the hospitals. Next, the patients submit the claim materials to insurance companies to make a claim application.

However, most of the patients do not know what materials are required for insurance claims after they seek medical treatment. It takes a lot of time for these patients to consult relevant personnel to determine the materials required for insurance claims, resulting in a poor experience for the patients.

### SUMMARY

Some embodiments of the present disclosure provide following technical solutions.

In a first aspect, there is provided a method for obtaining insurance claim materials, which is applied to a business server. The method includes:
sending the first query request to an insurance server;
the first query request comprising identification information of a target patient;
receiving insurance product information purchased by the target patient returned by the insurance server;
the insurance product information being obtained by querying by the insurance server according to the identification information;
comparing diagnosis and treatment information of the target patient with the insurance product information to determine whether a comparison result meets a claim prohibition condition;
determining a list of target claim materials based on the insurance product information and a list of designated claim materials prestored in the business server when the comparison result does not meet the claim prohibition condition;
obtaining, from a hospital server, target claim material information corresponding to the list of target claim materials, and sending the target claim material information to a terminal device; and
the target claim material information comprising an electronic document of the target claim materials and/or information for receiving the target claim materials.

In a second aspect, a business server is provided, including:
a first query request sending module, configured to send a first query request to an insurance server, the first query request comprising identification information of a target patient;
an insurance product information receiving module, configured to receive insurance product information purchased by the target patient returned by the insurance server;
the insurance product information being obtained by querying by the insurance server according to the identification information;
a claim condition determination module, configured to compare diagnosis and treatment information of the target patient with the insurance product information to determine whether a comparison result meets a claim prohibition condition;
a module for determining a list of target claim materials, configured to determine a list of target claim materials based on the insurance product information and a list of designated claim materials prestored in the business server when the comparison result does not meet the claim prohibition condition; and
a target claim material information sending module, configured to obtain, from a hospital server, target claim material information corresponding to the list of target claim materials, and send the target claim material information to a terminal device.

The target claim material information includes an electronic document of the target claim materials and/or information for receiving the target claim materials.

In a third aspect, there is provided a business server, which includes a processor, a memory, and a computer program stored in the memory and executed by the processor. The computer program is executable by the processor, whereby steps of the method for obtaining insurance claim materials are performed.

In a fourth aspect, there is provided a computer program, including a computer readable code. When the computer readable coder is operated on a business server, the business server executes the steps of the method for obtaining insurance claim materials.

In a fifth aspect, there is provided a computer-readable medium, storing a computer program. The computer program is executable by a processor, whereby steps of the method for obtaining insurance claim materials are performed.

In a sixth aspect, there is provided a system for obtaining insurance claim materials, which includes a terminal device, a hospital server, an insurance server, and the business server mentioned above.

The terminal device is configured to receive target claim material information corresponding to the list of target claim materials sent by the business server, a planned path for reception, and recommended reception time when the comparison result does not meet the claim prohibition condition.

The terminal device is further configured to: receive item information not meeting a claim condition sent by the business server when the comparison result meets the claim prohibition condition, to receive information on preset claim materials sent by the business server when the insurance server fails to search out the insurance product information purchased by the target patient, and to send a claim request to the business server.

The hospital server is configured to: send medical information of the target patient to the business server; send target claim material information corresponding to the list of target claim materials to the business server, or send information on preset claim materials corresponding to the list of preset claim materials to the business server; and receive a second query request sent by the business server, and return, to the business server according to the second query request, a two-dimensional code corresponding to an in-hospital navigation system or a hospital plan display view.

The insurance server is configured to: receive a first query request sent by the business server, and return, to the business server according to the first query request, the insurance product information purchased by the target patient or wrong information; and receive an electronic document of the target claim materials sent by the business server.

### BRIEF DESCRIPTION OF THE DRAWINGS

To describe the technical solutions in the embodiments of the present disclosure or the existing technologies more clearly, the accompanying drawings required for describing the embodiments or the existing technologies will be briefly introduced below. Apparently, the accompanying drawings in the following description are merely some embodiments of the present disclosure. To those of ordinary skills in the art, other accompanying drawings may also be derived from these accompanying drawings without creative efforts.

FIG. 1 schematically illustrates a flowchart of a method for obtaining insurance claim materials according to an embodiment of the present disclosure;

FIG. 2 schematically illustrates a flowchart of another method for obtaining insurance claim materials according to an embodiment of the present disclosure;

FIG. 3 schematically illustrates a schematic diagram of each field included in a field set file according to an embodiment of the present disclosure;

FIG. 4 schematically illustrates a schematic diagram of processing insurance product information based on a combination of target insurance fields and comparing the to-be-compared insurance product information processed with diagnosis and treatment information according to an embodiment of the present disclosure;

FIG. 5 schematically illustrates a structural block diagram of a business server according to an embodiment of the present disclosure;

FIG. 6 schematically illustrates a schematic diagram of a system for obtaining insurance claim materials according to an embodiment of the present disclosure;

FIG. 7 schematically illustrates a block diagram of a business server for performing the method according to the present disclosure; and

FIG. 8 schematically illustrates a memory cell for maintaining or carrying a program code for implementing the method according to the present disclosure.

### DETAILED DESCRIPTION

To make the objectives, technical solutions, and advantages of the embodiments of the application clearer, the technical solutions in the embodiments of the present disclosure will be described clearly and completely below with reference to the accompanying drawings in the embodiments of the present disclosure. Apparently, the described embodiments are some but not all of the embodiments of the present disclosure. All other embodiments obtained by a person of ordinary skill in the art based on the embodiments of the present disclosure without creative efforts shall fall within the protection scope of the present disclosure.

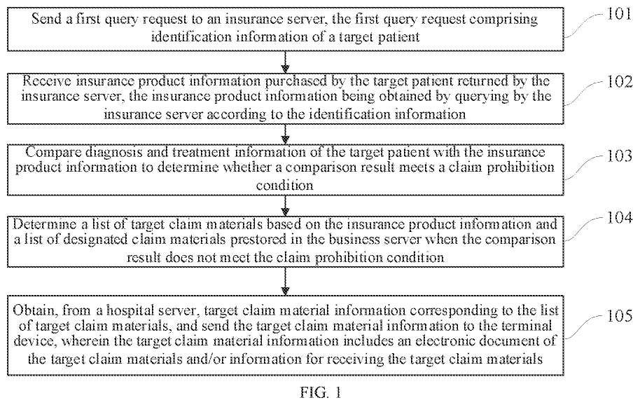

FIG. 1 schematically illustrates a flowchart of a method for obtaining insurance claim materials according to an embodiment of the present disclosure. This method may be applied to, for example, a business server. Specifically, this method may include following steps.

Step 101: sending a first query request to an insurance server, the first query request comprising identification information of a target patient.

In this embodiment of the present disclosure, when receiving a preset trigger operation, the business server sends the first query request to the insurance server, wherein the first query request includes the identification information of the target patient. The insurance server receives the first query request sent by the business server, and queries, according to the identification information in the first query request, whether the insurance server stores the insurance product information purchased by the target patient corresponding to the identification information. That is, it is determined whether the target patient has purchased insurance.

The identification information of the target patient includes an identity card number of the target patient, and the insurance server refers to a server corresponding to an insurance company.

For example, if an target patient A was injured in a car accident and was hospitalized, when receiving the preset trigger operation, the business server sends the first query request to the insurance server corresponding to an insurance company B.

Step 102: receiving insurance product information purchased by the target patient returned by the insurance server, the insurance product information being obtained by querying by the insurance server according to the identification information.

In this embodiment of the present disclosure, if the insurance server searches out that the insurance server stores the insurance product information purchased by the target patient corresponding to the identification information, the insurance server the insurance product information purchased by the target patient to the business server, and the business server receives the insurance product information purchased by the target patient returned by the insurance server.

For example, when the insurance server corresponding to the insurance company B searches out that the insurance server stores insurance product information purchased by the target patient A, the insurance server sends the insurance product information purchased by the target patient A to the business server.

Step 103: comparing diagnosis and treatment information of the target patient with the insurance product information to determine whether a comparison result meets a claim prohibition condition.

In this embodiment of the present disclosure, the business server obtains the diagnosis and treatment information of the target patient, and compares the diagnosis and treatment information of the target patient with the insurance product information purchased by the target patient to determine whether the comparison result meets the claim prohibition condition.

Step 104: determining a list of target claim materials based on the insurance product information and a list of designated claim materials prestored in the business server when the comparison result does not meet the claim prohibition condition.

In this embodiment of the present disclosure, when the comparison result does not meet the claim prohibition condition, that is, when the insurance product information purchased by the target patient does not meet the claim condition, the business server sends a first reminder message to the terminal device to remind the target patient to apply for insurance claim, and remind the target patient whether to obtain target claim material information. When the terminal device receives a first acknowledged operation from the target patient, the terminal device sends a request for obtaining the target claim material information to the business server, such that the business server determines a list of designated claim materials prestored in the business server according to the request for obtaining the target claim material information. The list of designated claim materials refers to a list of claim materials, stored in the business server, corresponding to an insurance type of the insurance product information. The list of designated claim materials includes a material name corresponding to at least one of the claim materials.

Next, the business server determines a list of target claim materials based on the insurance product information and the list of designated claim materials prestored in the business server. The list of target claim materials refers to a list of materials shared by the list of claim materials included in the insurance product information and the list of designated claim materials.

Step 105: obtaining, from a hospital server, target claim material information corresponding to the list of target claim materials, and sending the target claim material information to the terminal device, wherein the target claim material information includes an electronic document of the target claim materials and/or information for receiving the target claim materials.

In this embodiment of the present disclosure, after determining the list of target claim materials, the business server sends a first acquisition request to the hospital server, wherein the first acquisition request includes the list of target claim materials. The hospital server queries, according to the first acquisition request, target claim material information corresponding to the list of target claim materials that is prestored in the hospital server, wherein the target claim material information includes an electronic document of the target claim materials and/or information for receiving the target claim materials. Next, the hospital server sends the target claim material information corresponding to the list of target claim materials to the business server. The business server receives the target claim material information sent by the hospital server, and sends the target claim material information to the terminal device.

The target patient may view, on the terminal device, the electronic document of the target claim materials and/or the information for receiving the target claim materials. The target claim materials refer to claim materials issued by a hospital and required for applying for insurance claim by the target patient. The electronic document of the target claim materials refers to specific contents included in the target claim materials of an electronic version. The information for receiving the target claim materials refers to information required by the target patient to receive the target claim materials, such as a material name of the target claim materials, and a site for receiving the target claim materials, etc.

The target patient may view, on the terminal device, the electronic document of the target claim materials and/or the information for receiving the target claim materials. In this way, the target patient can timely know types of materials required for insurance claim, and does not need to spend a lot of time to consult relevant personnel, such that time can be saved for the target patient, and experience can be improved for the target patient.

When the business server sends the electronic document of the target claim materials to the terminal device, the target patient may directly make a claim application based on the electronic document of the target claim materials. When the business server sends the information for receiving the target claim materials to the terminal device, the target patient may also go to the hospital to receive the claim materials and then make the claim application according to the information for receiving the target claim materials.

It is to be noted that the terminal device may be a mobile terminal designated by the target patient, such as a mobile phone held by the target patient, and the target patient may directly view, on the mobile terminal, the target claim material information. The terminal device may be an electronic device deployed in the hospital, and the target patient may view the target claim material information corresponding to the target patient by operating the electronic device deployed in the hospital and entering the corresponding identification information.

In addition, the business server and the hospital server in this embodiment of the present disclosure may be different servers or may be the same server.

In an alternative implementation of the present disclosure, after Step 101, the method also includes Step S106, Step S107, Step S108, and Step S109.

Step S106: receiving wrong information returned by the insurance server, wherein the wrong information is generated after the insurance server fails to search out the insurance product information purchased by the target patient according to the identification information.

Step S107: determining a list of preset claim materials prestored in the business server.

Step S108: obtaining, from the hospital server, information on preset claim materials corresponding to the list of preset claim materials, wherein the information on preset claim materials comprises an electronic document of the preset claim materials and/or information for receiving the preset claim materials.

Step S109: sending the information on preset claim materials to the terminal device.

In this embodiment of the present disclosure, if the insurance server searches out that the insurance server does not store the insurance product information purchased by the target patient corresponding to the identification information, the insurance server generates wrong information and sends the wrong information to the business server, such that the business server receives the wrong information returned by the insurance server.

When the business server receives the wrong information returned by the insurance server, the business server determines that the target patient has not purchased the insurance, and the business server sends a second reminder message to the terminal device to remind the target patient that no insurance purchase record is searched out, and remind the target patient whether to obtain the information on preset claim materials. When the terminal device receives a second acknowledged operation from the target patient, the terminal device sends a request for obtaining the information on preset claim materials to the business server, such that the business server determines a list of preset claim materials prestored in the business server according to the request for obtaining the information on preset claim materials. The list of preset claim materials refers to a list of all claim materials stored in the business server.

Next, the business server sends a second acquisition request to the hospital server, wherein the second acquisition request includes the list of preset claim materials prestored in the business server. The hospital server queries, based on the second acquisition request, the information on preset claim materials corresponding to the list of preset claim materials prestored in the hospital server, and sends the information on preset claim materials to the business server. The information on preset claim materials includes the electronic document of the preset claim materials and/or the information for receiving the preset claim materials. The preset claim materials refer to all claim materials that can be issued by the hospital.

After receiving the information on preset claim materials returned from the hospital server, the business server sends the information on preset claim materials to the terminal device.

Thus, when the insurance server does not search out the insurance product information purchased by the target patient, the business server obtains, from the hospital server, electronic documents of all the claim materials that can be issued by the hospital and/or information for receiving all the claim materials that can be issued by the hospital, and sends them to the terminal device.

Alternatively, after step 103, the method further comprises: sending item information not meeting a claim condition to the terminal device when the comparison result meets the claim prohibition condition, wherein the item information comprises a name of the item not meeting the claim condition and a cause for not meeting the claim condition.

In this embodiment of the present disclosure, after the diagnosis and treatment information of the target patient is compared with the insurance product information, when the comparison result meets the claim prohibition condition, that is, when the insurance product information purchased by the target patient does not meet the claim condition, the item information not meeting the claim condition is sent to the terminal device, wherein the item information comprises the name of the item not meeting the claim condition and the cause for not meeting the claim condition.

For example, a scope of liability exemption information included in the insurance product information includes drunk driving, and a diagnosis result in the diagnosis and treatment information of the target patient A includes: compound trauma xx, and an alcohol content in a blood test result is overproof. In this case, after the insurance product information is compared with the diagnosis and treatment information of the target patient A, it is determined that the comparison result meets the claim prohibition condition, and the item information not meeting the claim condition is sent to the terminal device, wherein in the item information, the name of the item not meeting the claim condition is the diagnosis result, and the cause for not meeting the claim condition is that the diagnosis result falls within the scope of liability exemption information.

In this embodiment of the present disclosure, the diagnosis and treatment information of the target patient is compared with the insurance product information purchased by the target patient to determine whether the comparison result meets the claim prohibition condition. The list of target claim materials is determined based on the insurance product information and the list of designated claim materials prestored in the business server when the comparison result does not meet the claim prohibition condition, and the target claim material information corresponding to the list of target claim materials is sent to the terminal device, such that the target patient may view, on the terminal device, the electronic document of the target claim materials and/or the information for receiving the target claim materials. In this way, the target patient can timely know types of materials required for insurance claim, and does not need to spend a lot of time to consult relevant personnel, such that time can be saved for the target patient, and experience can be improved for the target patient.

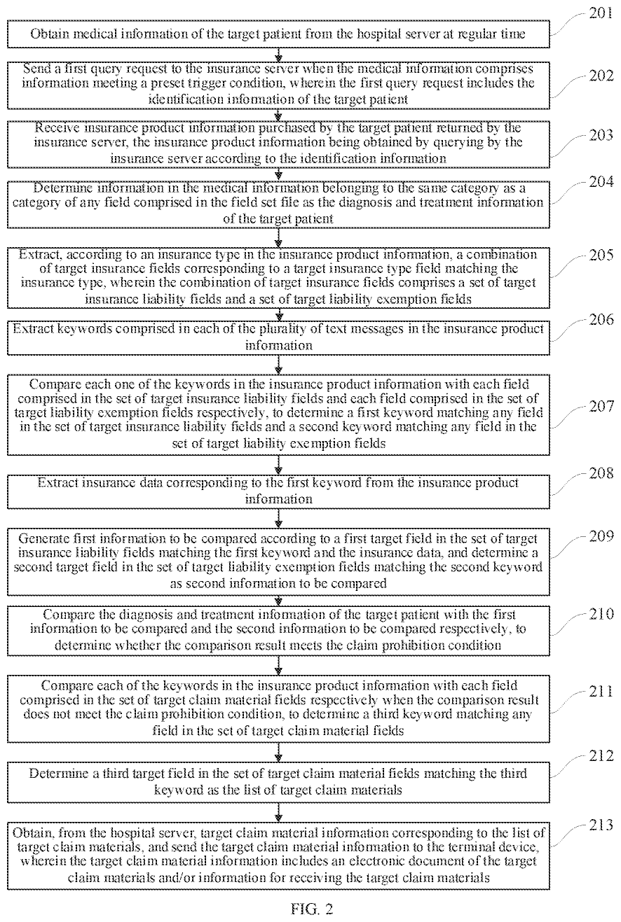

Referring to FIG. 2, FIG. 2 schematically illustrates a flowchart of another method for obtaining insurance claim materials according to an embodiment of the present disclosure. This method may be applied to a business server. Specifically, this method may include following steps.

Step 201: obtaining medical information of the target patient from the hospital server at regular time.

In this embodiment of the present disclosure, the business server sends a request for obtaining medical information to the hospital server at regular time, wherein the request for obtaining medical information includes identification information of the target patient. The hospital server queries the medical information of the target patient prestored in the hospital server based on the identification information in the request for obtaining medical information sent by the business server, and the hospital server sends the medical information of the target patient to the business server. In this way, the business server obtains the medical information of the target patient from the hospital server at regular time.

For example, the business server obtains the medical information of the target patient from the hospital server every two hours.

Step 202: sending a first query request to the insurance server when the medical information comprises information meeting a preset trigger condition, wherein the first query request includes the identification information of the target patient.

In this embodiment of the present disclosure, when the business server detects that the medical information of the target patient contains the information meeting the preset trigger condition, i.e., when the business server receives a preset trigger operation, the business server sends the first query request to the insurance server, wherein the first query request includes the identification information of the target patient. The insurance server receives the first query request from the business server, and queries, based on the identification information in the first query request, whether the insurance product information purchased by the target patient corresponding to the identification information is stored in the insurance server.

For example, the diagnosis result in the medical information of the target patient A is compared with critical disease information stored in the business server, and it is determined that the target patient A suffers from a critical disease. In this case, it is detected that the medical information of the target patient contains the information meeting the preset trigger condition; or if the medical information of the target patient includes a discharge instruction issued by a doctor, the business server determines that the medical information of the target patient contains the information meeting the preset trigger condition.

Step 203: receiving insurance product information purchased by the target patient returned by the insurance server, the insurance product information being obtained by querying by the insurance server according to the identification information.

This step is similar in principle to the Step 102, and thus is not unnecessarily elaborated herein.

Step 204: determining information in the medical information belonging to the same category as a category of any field comprised in the field set file as the diagnosis and treatment information of the target patient.

In this embodiment of the present disclosure, the field set file is constructed in advance based on basic personal information of a patient and the diagnosis and treatment information of the patient, and the field set file is stored into the business server. The field set file comprises at least one insurance type field, and a combination of insurance fields corresponding to each of the insurance type fields. The combination of insurance fields comprises a set of insurance liability fields, a set of liability exemption fields, and a set of claim material fields. The set of insurance liability fields comprises a subset of insurance time fields, a subset of insurance content fields, and a subset of compensation manner fields. The subset of insurance time fields comprises at least one insurance time field, the subset of insurance content fields comprises at least one insurance content field, and the subset of compensation manner fields comprises at least one compensation manner field. The set of liability exemption fields comprises at least one liability exemption field, and the set of claim material fields comprises at least one claim material field.

Maintenance costs for subsequent maintenance of the field set file can be reduced by dividing the field set file into a set of insurance type fields, the set of insurance liability fields, the set of liability exemption fields, and the set of claim material fields according to different field types.

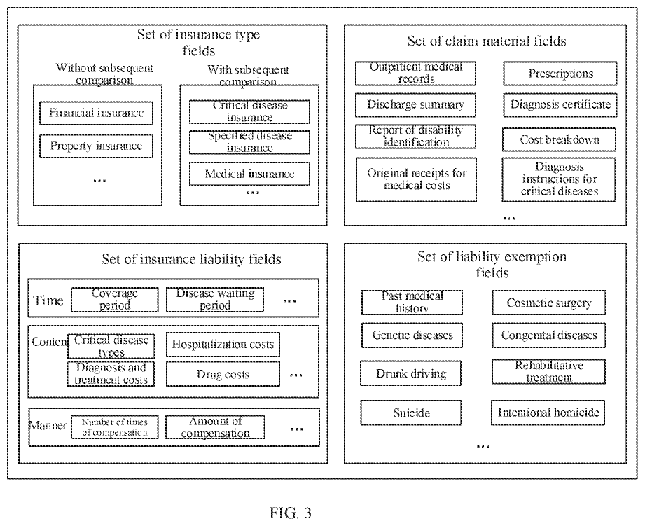

As shown in FIG. 3, the set of insurance type fields in the field set file is divided into a subset of fields without subsequent comparison and a subset of fields with subsequent comparison. The subset of fields without subsequent comparison includes fields such as financial insurance and property insurance. The subset of fields with subsequent comparison includes fields such as critical disease insurance, specified disease insurance, and medical insurance, wherein the critical disease insurance, the specified disease insurance and the medical insurance are all insurance type fields included in the field set file.

The set of insurance liability fields in the field set file includes the subset of insurance time fields, the subset of insurance content fields, and the subset of compensation manner fields. The subset of insurance time fields includes at least one insurance time field, for example, time-related fields such as a coverage period and a disease waiting period. The subset of insurance content fields includes at least one insurance content field, for example, an insurance coverage field such as critical disease type, hospitalization costs, diagnosis and treatment costs, and drug costs. The subset of compensation manner fields includes at least one compensation manner field, for example, a claim manner field such as number of times of compensation and amount of compensation.

The set of liability exemption fields in the field set file includes at least one liability exemption field, for example, a field not meeting a claim condition such as past medical history, genetic diseases, drunk driving, suicide, cosmetic surgery, congenital diseases, rehabilitative treatment, and intentional homicide.

The set of claim material fields in the field set file includes at least one claim material field, for example, outpatient medical records, discharge summary, report of disability identification, original receipts for medical costs, prescriptions, diagnosis certificate, cost breakdown and diagnosis instructions for critical diseases, and other claim material fields that can be issued by the hospital.

Different mapping relationships exist between each insurance type field in the set of insurance type fields and the set of insurance liability fields, the set of liability exemption fields, and the set of claim material fields. Thus, after the corresponding insurance type fields are determined based on the insurance product information, the set of insurance liability fields, the set of liability exemption fields and the set of claim material fields with the mapping relationships can be determined.

Each time after obtaining the medical information of the target patient, the business server determines a category to which each piece of information in the medical information of the target patient belongs, determines information in the medical information belonging to the same category as the category of any field comprised in the field set file as the diagnosis and treatment information of the target patient, and stores the diagnosis and treatment information in the business server for subsequent comparison with the insurance product information.

For example, the coverage period and disease waiting period both belong to time-related information. Therefore, the time-related information in the medical information is extracted to obtain the diagnosis and treatment information of the target patient, that is, hospitalization time 2020-02-12 and the discharge time 2020-04-15 in the medical information are extracted. The drug costs belong to drug-related information, so the drug-related information in the medical information is extracted to obtain the diagnosis and treatment information of the target patient, that is, drug items xxx in the medical information and costs corresponding to each of the drug items or the like are extracted.

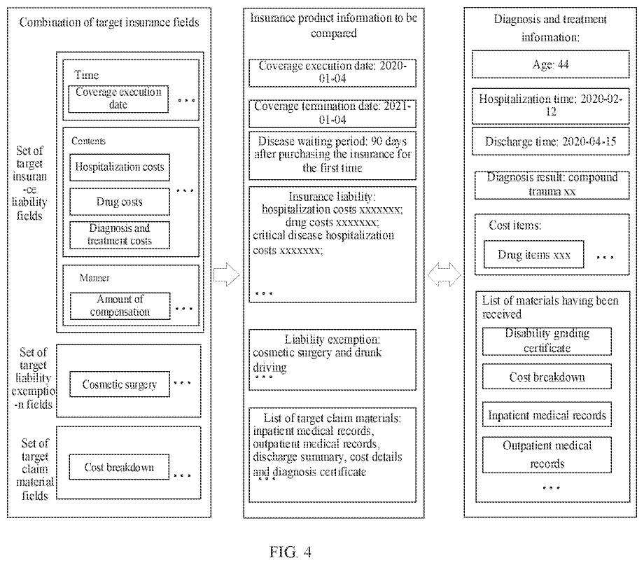

According to the above method, the information belonging to the same category as the category of any field included in the field set file is extracted from the medical information to obtain the diagnosis and treatment information of the target patient. As shown in FIG. 4, the diagnosis and treatment information of the target patient includes: age (44) of the target patient A, hospitalization time 2020-02-12) of the target patient A, the discharge time 2020-04-15) of the target patient A, and diagnosis result (compound trauma xx). The cost items include the drug items xxx and the costs corresponding to each of the drug items or the like, and a list of materials having been received by target patient A, including: disability grading certificate, cost breakdown, inpatient medical records, and outpatient medical records, etc.

Step 205: extracting, according to an insurance type in the insurance product information, a combination of target insurance fields corresponding to a target insurance type field matching the insurance type, wherein the combination of target insurance fields comprises a set of target insurance liability fields and a set of target liability exemption fields.

In this embodiment of the present disclosure, after receiving the insurance product information purchased by the target patient returned by the insurance server, an insurance type in the insurance product information is obtained, and the insurance type in the insurance product information is compared with each insurance type field included in the set of insurance type fields in the field set file, to determine a target insurance type field matching the insurance type. The target insurance type field is one insurance type field in the set of insurance type fields.

Next, a set of target insurance liability fields and a set of target liability exemption fields corresponding to the target insurance type field are extracted according to different mapping relationships between each insurance type field and the set of insurance liability fields, the set of liability exemption fields, and the set of claim material fields. The set of target insurance liability fields refers to a set of insurance liability fields corresponding to the target insurance type fields in the set of insurance liability fields included in the field set file. The set of target liability exemption fields refers to a set of liability exemption fields corresponding to the target insurance type fields in the set of liability exemption fields included in the field set file.

For example, if the target insurance type field is medical insurance, as shown in FIG. 4, the combination of target insurance fields corresponding to the target insurance type field includes the set of target insurance liability fields and the set of target liability exemption fields. The set of target insurance liability fields comprises a subset of target insurance time fields, a subset of target insurance content fields, and a subset of target compensation manner fields. The subset of target insurance time fields comprises at least one target insurance time field, for example, a field such as coverage execution date. The subset of target insurance content fields comprises at least one target insurance content field, for example, a field such as hospitalization costs, drug costs, and diagnosis and treatment costs. The subset of target compensation manner fields comprises at least one target compensation manner field, for example, a field such as amount of compensation. The set of target liability exemption fields comprises at least one target liability exemption field, for example, a field such as cosmetic surgery.

Step 206: extracting keywords comprised in each of the plurality of text messages in the insurance product information.

In this embodiment of the present disclosure, after the insurance product information returned by the insurance server is received, the insurance product information is split into a plurality of text messages, wherein each of the plurality of text messages may be a complete sentence to be processed for word segmentation. Next, the keywords included in each of the plurality of text messages are extracted.

For example, the keywords extracted from the insurance product information include “execution time”, and “cosmetic surgery”, etc.

Step 207: comparing each one of the keywords in the insurance product information with each field comprised in the set of target insurance liability fields and each field comprised in the set of target liability exemption fields respectively, to determine a first keyword matching any field in the set of target insurance liability fields and a second keyword matching any field in the set of target liability exemption fields.

In this embodiment of the present disclosure, after the keywords included in each of the plurality of text messages in the insurance product information are extracted, each one of the keywords in the insurance product information is compared with each field comprised in the set of target insurance liability fields and each field comprised in the set of target liability exemption fields respectively. That is, a similarity between each one of the keywords in the insurance product information and each field comprised in the set of target insurance liability fields and each field comprised in the set of target liability exemption fields. When the similarity is greater than a threshold, it is determined that the keyword matches the field, and a keyword matching any field in the set of target insurance liability fields is determined as the first keyword, and a keyword matching any field in the set of target liability exemption fields is determined as the second keyword.

For example, a keyword in the insurance product information is “execution date”, and it matches the field “coverage execution date” in the set of target insurance liability fields. In this case, the keyword “execution date” is determined as the first keyword. For another example, a keyword in the insurance product information is “cosmetic surgery”, and it matches the field “cosmetic surgery” in the set of target liability exemption fields. In this case, the keyword “cosmetic surgery” is determined as the second keyword.

Step 208: extracting insurance data corresponding to the first keyword from the insurance product information.

In this embodiment of the present disclosure, after the first keyword matching any field in the set of target insurance liability fields is determined, insurance data corresponding to each first keyword is extracted from the insurance product information.

For example, the first keyword is “execution time”, and the insurance data corresponding to the first keyword “execution time” extracted from the insurance product information is 2020-01-04.

Step 209: generating first information to be compared according to a first target field in the set of target insurance liability fields matching the first keyword and the insurance data, and determining a second target field in the set of target liability exemption fields matching the second keyword as second information to be compared.

In this embodiment of the present disclosure, after the insurance data corresponding to each first keyword are extracted, the first information to be compared is generated according to the first target field in the set of target insurance liability fields matching the first keyword and the insurance data corresponding to the first keyword. That is, the first information to be compared having a specific format is generated by writing the insurance data after the first target field.

The second target field in the set of target liability exemption fields matching the second keyword is directly determined as the second information to be compared. That is, the second keyword in the insurance product information is converted into the second target field, to compare the insurance product information with the diagnosis and treatment information of the target patient by means of the second target field.

For example, the first target field matching the first keyword “execution time” is “coverage execution date”, and the insurance data corresponding to the first keyword “execution time” is 2020-01-04. In this case, the generated first information to be compared includes the “coverage execution date” 2020-01-04. If the second keyword “cosmetic surgery” matches the second target field “cosmetic surgery”, the second target field “cosmetic surgery” is determined as the second information to be compared.

According to the above method, corresponding information may be extracted from the insurance product information based on the set of target insurance liability fields and the set of target liability exemption fields, and the insurance product information to be compared as shown in FIG. 4 is generated, wherein the insurance product information to be compared includes the first information to be compared and the second information to be compared. The first information to be compared includes: “coverage execution date” 2020-01-04, “coverage termination date” 2021-01-04, “disease waiting period” 90 days after purchasing the insurance for the first time, hospitalization costs xxxxxxx, drug costs xxxxxxx, and critical disease hospitalization costs xxxxxxx. The second information to be compared includes: cosmetic surgery and drunk driving.

A field set file is stored in the business server, the set of target insurance liability fields and the set of target liability exemption fields in the field set file are determined based on the insurance type in the insurance product information purchased by the target patient, and the corresponding information is extracted from the insurance product information to generate the first information to be compared and the second information to be compared based on the set of target insurance liability fields and the set of target liability exemption fields. In this way, healthcare-related information to be compared in the insurance product information is retained, and some unnecessary information is removed, to facilitate subsequent comparison between the diagnosis and treatment information of the target patient and the first information to be compared and the second information to be compared, thereby reducing the amount of data for comparison.

Step 210: comparing the diagnosis and treatment information of the target patient with the first information to be compared and the second information to be compared respectively, to determine whether the comparison result meets the claim prohibition condition.

In this embodiment of the present disclosure, after the diagnosis and treatment information of the target patient and the first information to be compared and the second information to be compared are obtained, the diagnosis and treatment information is respectively compared with the first information to be compared and the second information to be compared, to determine whether the comparison result meets the claim prohibition condition.

Specifically, the Step 210 includes Substep S2101, Substep S2102, and Substep S2103.

Substep S2101: comparing information belonging to a same category in the first information to be compared and the diagnosis and treatment information one by one, to determine whether there exists information matching the second information to be compared in the diagnosis and treatment information.

Substep S2102: determining that the comparison result does not meet the claim prohibition condition when the information belonging to the same category matches and there does not exist the information matching the second information to be compared in the diagnosis and treatment information.

Substep S2103: determining that the comparison result meets the claim prohibition condition when the information belonging to the same category does not match and there exists the information matching the second information to be compared in the diagnosis and treatment information.

In this embodiment of the present disclosure, a category of each piece of information in the first information to be compared and the diagnosis and treatment information is first determined, then information belonging to the same category in the first information to be compared and the diagnosis and treatment information is compared one by one, to determine whether there exists information matching the second information to be compared in the diagnosis and treatment information.

It is determined that the comparison result does not meet the claim prohibition condition when the information belonging to the same category in the first information to be compared and the diagnosis and treatment information matches and there does not exist the information matching the second information to be compared in the diagnosis and treatment information.

However, it is determined that the comparison result meets the claim prohibition condition when the information belonging to the same category in the first information to be compared and the diagnosis and treatment information does not match and there exists the information matching the second information to be compared in the diagnosis and treatment information. Specifically, it is determined that the comparison result meets the claim prohibition condition when the information belonging to the same category in the first information to be compared and the diagnosis and treatment information does not match. It is determined that the comparison result meets the claim prohibition condition when there exists the information matching the second information to be compared in the diagnosis and treatment information. It is determined that the comparison result meets the claim prohibition condition when the information belonging to the same category in the first information to be compared and the diagnosis and treatment information does not match and there exists the information matching the second information to be compared in the diagnosis and treatment information.

For example, hospitalization time 2020-02-12 and discharge time 2020-04-15 in the diagnosis and treatment information both belong to time-related information, and coverage execution time 2020-01-04 and coverage termination time 2021-01-04 in the first information to be compared both belong to time-related information. The hospitalization time and the discharge time are compared with the coverage execution time and the coverage termination time to determine a time period between the hospitalization time and the discharge time is after the coverage execution time and before the coverage termination time, that is, it is determined that the information belonging to the same category in the first information to be compared and the diagnosis and treatment information matches. Accordingly, other information belonging to the same category also matches, and there is no information matching “drunk driving” and “cosmetic surgery” in the diagnosis and treatment information. In this case, it is determined that the comparison result does not meet the claim prohibition condition.

Step 211: comparing each of the keywords in the insurance product information with each field comprised in the set of target claim material fields respectively when the comparison result does not meet the claim prohibition condition, to determine a third keyword matching any field in the set of target claim material fields.

In this embodiment of the present disclosure, the combination of target insurance fields also includes the set of target claim material fields. When the Step 205 is performed, it is also extracted, according to an insurance type in the insurance product information, the set of target claim material fields corresponding to a target insurance type field matching the insurance type. The set of target claim material fields refers to a set of claim material fields corresponding to the target insurance type fields in the set of claim material fields included in the field set file.

As shown in FIG. 4, the combination of target insurance fields also includes a set of target claim material fields, wherein the set of target claim material fields includes at least one claim material field, such as cost breakdown.

When the comparison result does not meet the claim prohibition condition, the business server sends a first reminder message to the terminal device to remind the target patient to apply for insurance claim, and remind the target patient whether to obtain target claim material information. When the terminal device receives a first acknowledged operation from the target patient, the terminal device sends a request for obtaining the target claim material information to the business server, such that the business server compares, based on the request for obtaining the target claim material information sent by the terminal device, each of the keywords in the insurance product information with each field comprised in the set of target claim material fields respectively, to calculate a similarity between each of the keywords and each field comprised in the set of target claim material fields. When the similarity is greater than a threshold, it is determined that the keyword matches the field, and a keyword matching any field in the set of target claim material fields is determined as the third keyword.

For example, if a keyword in the insurance product information is “cost details”, and this keyword matches the field “cost breakdown” in the set of target claim material fields, the keyword “cost details” is determined as the third keyword.

It is to be noted that the list of designated claim materials in the above Embodiment I also refers to each target claim material field included in the set of target claim material fields.

Step 212: determining a third target field in the set of target claim material fields matching the third keyword as the list of target claim materials.

In this embodiment of the present disclosure, after the third keyword matching any field in the set of target claim material fields is determined, the third target field matching the third keyword in the set of target claim material fields is directly used as the list of target claim materials, that is, the third keyword in the insurance product information is converted into the third target field.

For example, if the third target field matching the third keyword “cost details” is “cost breakdown”, the third target field “cost breakdown” is determined as the list of target claim materials.

According to the above method, the third keyword may be extracted from the insurance product information based on the set of target claim material fields, and the third keyword is converted into the third target field, to generate the list of target claim materials in the insurance product information to be compared as shown in FIG. 4. The list of target claim materials includes: inpatient medical records, outpatient medical records, discharge summary, cost details and diagnosis certificate.

Step 213: obtaining, from a hospital server, target claim material information corresponding to the list of target claim materials, and sending the target claim material information to the terminal device, wherein the target claim material information includes an electronic document of the target claim materials and/or information for receiving the target claim materials.

This step is similar in principle to the Step 105, and thus is not unnecessarily elaborated herein.

Specifically, the diagnosis and treatment information of the target patient includes a list of materials having been received. The Step 213 includes Substep S2131, Substep S2132, and Substep S2133.

Substep S2131: determining a list of materials not matching the list of materials having been received from the list of target claim materials as a list of materials having not been received.

Substep S2132: obtaining, from the hospital server, an electronic document of materials having been received corresponding to the list of materials having been received, an electronic document of materials having not been received corresponding to the list of materials having not been received, and information for receiving the materials having not been received.

Substep S2133: sending, to the terminal device, the electronic document of materials having been received and/or information for receiving the materials having been received, and the electronic document of materials having not been received and/or the information for receiving the materials having not been received.

In this embodiment of the present disclosure, after determining the list of target claim materials, the business server compares the list of target claim materials with the list of materials having been received included in the diagnosis and treatment information one by one, and determines the list of materials not matching the list of materials having been received as the list of materials having not been received.

Next, the business server sends a first acquisition request to the hospital server, wherein the first acquisition request specifically includes the list of materials having been received and the list of materials having not been received. The hospital server queries, according to the first acquisition request, an electronic document of materials having been received corresponding to the list of materials having been received, an electronic document of materials having not been received corresponding to the list of materials having not been received, and information for receiving the materials having not been received. Next, the hospital server sends, to the business server, the electronic document of materials having been received corresponding to the list of materials having been received, an electronic document of materials having not been received corresponding to the list of materials having not been received, and the information for receiving the materials having not been received.

Next, the business server sends, to the terminal device, the electronic document of materials having been received and/or the information for receiving the materials having been received, and the electronic document of materials having not been received and/or the information for receiving the materials having not been received. Specifically, the business server may send, to the terminal device, the electronic document of materials having been received and the electronic document of materials having not been received. The business server may also send, to the terminal device, the information for receiving the materials having been received and the information for receiving the materials having not been received. The business server may also send, to the terminal device, the electronic document of materials having been received and the information for receiving the materials having not been received, etc.

The information for receiving the materials having been received includes: a material name of the materials having been received and first marking information of the materials having been received, wherein the first marking information is configured for indicating that the materials having been received have been received. The information for receiving the materials having not been received includes a material name of the materials having not been received, second marking information of the materials having not been received, and time, sites and attentions of receiving the materials having not been received. The second marking information is configured for indicating that the materials having not been received have not been received.

As shown in FIG. 4, the list of materials having been received includes disability grading certificate, cost breakdown, inpatient medical records, and outpatient medical records; and the list of target claim materials includes inpatient medical records, outpatient medical records, discharge summary, cost breakdown, and diagnosis certificate. In this case, it is determined that the list of materials having not been received includes discharge summary and diagnosis certificate. Next, the business server obtains the electronic document of materials having been received corresponding to the list of materials having been received, an electronic document of materials having not been received corresponding to the list of materials having not been received, and information for receiving the materials having not been received. Next, the business server sends, to the terminal device, the electronic document of materials having been received and/or the information for receiving the materials having been received, and the electronic document of materials having not been received and/or the information for receiving the materials having not been received.

Of course, in the actual use, the material names of the claim materials that can be issued by the hospital include: outpatient medical records, prescriptions, discharge summary, diagnosis certificate, original receipts for medical costs, cost breakdown during hospitalization, application form for extended hospitalization, diagnosis instructions for critical diseases, and report of disability identification, etc. Attentions of receiving the target claim materials refer to contents that need to be noted when receiving the target claim materials. For example, if it is needed to receive invoice information for prescriptions, when the business server sends the target claim materials including the prescriptions to the terminal device, the business server also sends the attentions of receiving the target claim materials to the terminal device accordingly, to remind the target patient to determine whether to exchange the current prescriptions for invoices.

It is to be noted that when comparing the diagnosis and treatment information of the target patient with the first information to be compared and the second information to be compared, it is determined that the comparison result does not meet the claim prohibition condition, but the diagnosis and treatment information includes medical projects or drug types that are not covered by the claim. In this case, when sending the target claim material information, the business server may accordingly send a prompt message to the terminal device, to remind the target patient of the medical projects or drug types that are not covered by the claim.

Further, after Step 213, Step S31 and Step S32 are also included.

Step S31: generating a planned path for reception according to a target location of the target patient and the site of receiving the materials having not been received.

Step S32: sending the planned path for reception to the terminal device.

In this embodiment of the present disclosure, when sending the target claim material information to the terminal device, the business server also sends a third reminder message to the terminal device accordingly, to remind the target patient whether to view the planned path for receiving the materials having not been received. When the terminal device receives a third acknowledged operation from the target patient, the terminal device sends a request for obtaining the planned path for reception to the business server. The business server receives the request for obtaining the planned path for reception sent by the terminal device, and generates the planned path for reception according to the target location of the target patient and the site of receiving the materials having not been received. The site of receiving the materials having not been received specifically includes information such as building number, floor, and room number.

The target location of the target patient may refer to a current location of the target patient or a ward location of the target patient during hospitalization. If the target location of the target patient is the current location of the target patient, when the terminal device sends the request for obtaining the planned path for reception to the business server, the request for obtaining the planned path for reception may carry information on the current location of the target patient. If the target location of the target patient is the ward location of the target patient during hospitalization, the business server may extract the ward location from the medical information of the target patient.

After the planned path for reception is generated, the business server sends the planned path for reception to the terminal device. The target patient may view, on the terminal device, the planned path for receiving the materials having not been received, and may receive the corresponding materials having not been received based on this planned path for reception. Each of the materials having not been received corresponds to a planned path for reception.

As mention above, the business server generates the planned path for reception and sends the planned path for reception to the terminal device. In this way, more high-quality and humanized services can be provided for the target patient, and service quality and medical experience can be improved for the target patient.

Specifically, Step S32 includes Substep S321, Substep S322, Substep S323, Substep S324, and Substep S325.

Substep S321: sending a second query request to the hospital server.

Substep S322: receiving a two-dimensional code corresponding to an in-hospital navigation system returned by the hospital server, and adding the planned path for reception into the two-dimensional code. The two-dimensional code is obtained after the hospital server searches out that a hospital is provided with the in-hospital navigation system according to the second query request.

Substep S323: sending, to the terminal device, the two-dimensional code with the planned path for reception added.

Substep S324: receiving a hospital plan display view returned by the hospital server, and marking the planned path for reception in the hospital plan display view. The hospital plan display view is obtained after the hospital server searches out that the hospital is provided with the in-hospital navigation system according to the second query request.

Substep S325: sending, to the terminal device, the hospital plan display view with the planned path for reception marked.

In this embodiment of the present disclosure, after generating the planned path for reception, the business server sends a second query request to the hospital server. The hospital server queries, according to the second query request, whether a hospital corresponding to the hospital server has an in-hospital navigation system. If the hospital has the in-hospital navigation system, the hospital server obtains a two-dimensional code corresponding to the in-hospital navigation system, and sends, to the business server, the two-dimensional code corresponding to the in-hospital navigation system. The business server receives the two-dimensional code corresponding to the in-hospital navigation system returned by the hospital server. Furthermore, the business server adds the planned path for reception into the two-dimensional code corresponding to the in-hospital navigation system, and then sends, to the terminal device, the two-dimensional code with the planned path for reception added.

If the terminal device is a mobile terminal designated by the target patient and the target patient recognizes the two-dimensional code with the planned path for reception added by means of the mobile terminal, the mobile terminal is connected to the in-hospital navigation system. If the terminal device is an electronic device deployed in the hospital and the target patient can scan, by means of the mobile terminal, the two-dimensional code displayed on the terminal device, the mobile terminal is connected to the in-hospital navigation system. After the mobile terminal is connected to the in-hospital navigation system, the planned path for reception of each of the materials having not been received may be displayed on a page of the mobile terminal, and the corresponding materials having not been received may be received based on the planned path for reception displayed.

However, if based on the second query request, the hospital server does not search out that the hospital has the in-hospital navigation system, the hospital server obtains a hospital plan display view, and returns the hospital plan display view to the business server. The business server receives the hospital plan display view returned by the hospital server, and marks the planned path for reception in the hospital plan display view, and then sends, to the terminal device, the hospital plan display view with the planned path for reception marked. Furthermore, the business server may also send, to the terminal device, text navigation information corresponding to the planned path for reception.

After the target patient receives the corresponding materials having not been received according to the two-dimensional code with the planned path for reception added or the hospital plan display view with the planned path for reception marked, the target patient performs a reception acknowledged operation on the terminal device. The terminal device sends information on receiving corresponding materials to the business server, and the business server saves reception records of the corresponding material.

Further, after Step 213, Step S33 and Step S34 are also included.

Step S33: generating recommended reception time according to the time of receiving the materials having not been received and target time corresponding to the target patient, wherein the target time comprises treatment time and/or hospital discharge time.

Step S34: sending the recommended reception time to the terminal device.

In this embodiment of the present disclosure, when sending the target claim material information to the terminal device, the business server also accordingly sends a fourth reminder message to the terminal device, to remind the target patient whether to view the recommended reception time of receiving the materials having not been received. When the terminal device receives a fourth acknowledged operation from the target patient, the terminal device sends, to the business server, a request for obtaining the recommended reception time. The business server receives the request for obtaining the recommended reception time sent by the terminal device, and then extracts, from the medical information of the target patient, target time corresponding to the target patient, wherein the target time comprises treatment time and/or hospital discharge time. Next, The business server generates the recommended reception time according to the time of receiving the materials having not been received and the target time corresponding to the target patient.

After the recommended reception time is generated, the business server sends the recommended reception time to the terminal device. The target patient may view, on the terminal device, the recommended reception time of receiving the materials having not been received, and may receive the corresponding materials having not been received based on the recommended reception time. In this way, reception efficiency can be improved for the target patient, because the target patient does not need to go to receive the materials for many times.

Further, after Step 213, Step S35 and Step S36 are also included.

Step S35: receiving a claim request sent by the terminal device.

Step S36: sending the electronic document of the target claim materials to the insurance server according to the claim request to make a claim application.

In this embodiment of the present disclosure, when sending the target claim material information to the terminal device, the business server also accordingly sends a fifth reminder message to the terminal device, to remind the target patient whether to upload the electronic document of the target claim materials to the insurance server to make a claim application. When the terminal device receives a fifth acknowledged operation from the target patient, the terminal device sends a claim request to the business server. The business server receives the claim request sent by the terminal device, and sends the electronic document of the target claim materials to the insurance server to make a claim application based on the claim request.

Through a claim material uploading portal provided by the insurance server corresponding to the insurance company, the target patient can one-click upload the electronic document of the claim materials to the insurance server by means of the terminal device and the business server, such that costs of consumables are reduced, and time waiting for reception is reduced.

In this embodiment of the present disclosure, the diagnosis and treatment information of the target patient is compared with the insurance product information purchased by the target patient to determine whether the comparison result meets the claim prohibition condition. The list of target claim materials is determined based on the insurance product information and the list of designated claim materials prestored in the business server when the comparison result does not meet the claim prohibition condition, and the target claim material information corresponding to the list of target claim materials is sent to the terminal device, such that the target patient may view, on the terminal device, the electronic document of the target claim materials and/or the information for receiving the target claim materials. In this way, the target patient can timely know types of materials required for insurance claim, and does not need to spend a lot of time to consult relevant personnel, such that time can be saved for the target patient, and experience can be improved for the target patient.

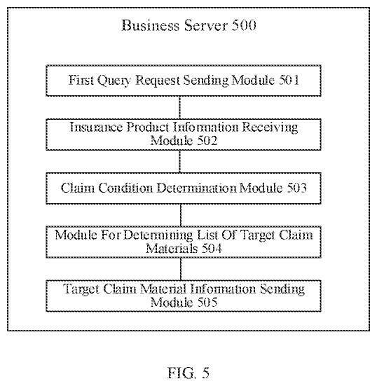

Referring to FIG. 5, FIG. 5 schematically illustrates a structural block diagram of a business server according to an embodiment of the present disclosure.

The business server 500 in this embodiment of the present disclosure includes:
a first query request sending module 501, configured to send a first query request to an insurance server, the first query request comprising identification information of a target patient;
an insurance product information receiving module 502, configured to receive insurance product information purchased by the target patient returned by the insurance server, the insurance product information being obtained by querying by the insurance server according to the identification information;
a claim condition determination module 503, configured to compare diagnosis and treatment information of the target patient with the insurance product information to determine whether a comparison result meets a claim prohibition condition;
a module 504 for determining a list of target claim materials, configured to determine a list of target claim materials based on the insurance product information and a list of designated claim materials prestored in the business server when the comparison result does not meet the claim prohibition condition; and
a target claim material information sending module 505, configured to obtain, from a hospital server, target claim material information corresponding to the list of target claim materials, and send the target claim material information to a terminal device. The target claim material information comprises an electronic document of the target claim materials and/or information for receiving the target claim materials.

Alternatively, a field set file is stored in the business server, wherein the field set file comprises at least one insurance type field, and a combination of insurance fields corresponding to each of the insurance type fields. The combination of insurance fields comprises a set of insurance liability fields, a set of liability exemption fields, and a set of claim material fields.

The set of insurance liability fields comprises a subset of insurance time fields, a subset of insurance content fields, and a subset of compensation manner fields. The subset of insurance time fields comprises at least one insurance time field, the subset of insurance content fields comprises at least one insurance content field, and the subset of compensation manner fields comprises at least one compensation manner field.

The set of liability exemption fields comprises at least one liability exemption field, and the set of claim material fields comprises at least one claim material field.

Alternatively, the claim condition determination module 503 includes:
a submodule for extracting a combination of target insurance fields, configured to extract, according to an insurance type in the insurance product information, a combination of target insurance fields corresponding to a target insurance type field matching the insurance type, wherein the combination of target insurance fields comprises a set of target insurance liability fields and a set of target liability exemption fields;
a keyword extracting submodule, configured to extract keywords comprised in each of the plurality of text messages in the insurance product information;
a keyword comparing submodule, configured to compare each one of the keywords in the insurance product information with each field comprised in the set of target insurance liability fields and each field comprised in the set of target liability exemption fields respectively, to determine a first keyword matching any field in the set of target insurance liability fields and a second keyword matching any field in the set of target liability exemption fields;
an insurance data extracting submodule, configured to extract insurance data corresponding to the first keyword from the insurance product information;
a submodule for generating information to be compared, configured to generate first information to be compared according to a first target field in the set of target insurance liability fields matching the first keyword and the insurance data, and determine a second target field in the set of target liability exemption fields matching the second keyword as second information to be compared; and
a claim condition determination submodule, configured to compare the diagnosis and treatment information of the target patient with the first information to be compared and the second information to be compared respectively, to determine whether the comparison result meets the claim prohibition condition.

Alternatively, the claim condition determination submodule includes:
an information comparing unit, configured to compare information belonging to a same category in the first information to be compared and the diagnosis and treatment information one by one, to determine whether there exists information matching the second information to be compared in the diagnosis and treatment information;
a first claim condition determination unit, configured to determine that the comparison result does not meet the claim prohibition condition when the information belonging to the same category matches, and there does not exist the information matching the second information to be compared in the diagnosis and treatment information; and
a second claim condition determination unit, configured to determine that the comparison result meets the claim prohibition condition when the information belonging to the same category does not match, and/or there exists the information matching the second information to be compared in the diagnosis and treatment information.

Alternatively, the combination of target insurance fields includes a set of target claim material fields, and the module 504 for determining a list of target claim materials includes:
a submodule for determining a third keyword, configured to compare each of the keywords in the insurance product information with each field comprised in the set of target claim material fields respectively when the comparison result does not meet the claim prohibition condition, to determine a third keyword matching any field in the set of target claim material fields; and
a submodule for determining the list of target claim materials, configured to determine a third target field in the set of target claim material fields matching the third keyword as the list of target claim materials.

Alternatively, the business server 500 also includes:
a medical information obtaining module, configured to obtain medical information of the target patient from the hospital server at regular time.
The medical information obtaining module is also configured to execute the first query request sending module 501 when the medical information comprises information meeting a preset trigger condition.

Alternatively, the business server 500 also includes:
a diagnosis and treatment information determining module, configured to determine information in the medical information belonging to a same category as a category of any field comprised in the field set file as the diagnosis and treatment information of the target patient.

Alternatively, the diagnosis and treatment information of the target patient comprises a list of materials having been received. The target claim material information sending module 505 includes:
a submodule for determining a list of materials having not been received, configured to determine a list of materials not matching the list of materials having been received from the list of target claim materials as the list of materials having not been received;
a target claim material information obtaining submodule, configured to obtain, from the hospital server, an electronic document of materials having been received corresponding to the list of materials having been received, an electronic document of materials having not been received corresponding to the list of materials having not been received, and information for receiving the materials having not been received; and
a target claim material information sending submodule, configured to send, to the terminal device, the electronic document of materials having been received and/or information for receiving the materials having been received, and the electronic document of materials having not been received and/or the information for receiving the materials having not been received.

The information for receiving the materials having been received includes a material name of the materials having been received and first marking information of the materials having been received, wherein the first marking information is configured for indicating that the materials having been received have been received. The information for receiving the materials having not been received includes a material name of the materials having not been received, second marking information of the materials having not been received, and time, sites and attentions of receiving the materials having not been received, wherein the second marking information is configured for indicating that the materials having not been received have not been received.

Alternatively, the business server 500 also includes:
a module for generating a planned path for reception, configured to generate a planned path for reception according to a target location of the target patient and the site of receiving the materials having not been received; and
a module for sending the planned path for reception, configured to send the planned path for reception to the terminal device.

Alternatively, the module for sending the planned path for reception includes:
a second query request sending submodule, configured to send a second query request to the hospital server;
a submodule for adding the planned path for reception, configured to receive a two-dimensional code corresponding to an in-hospital navigation system returned by the hospital server, and add the planned path for reception into the two-dimensional code, wherein the two-dimensional code is obtained after the hospital server searches out that a hospital is provided with the in-hospital navigation system according to the second query request;
a two-dimensional code sending submodule, configured to send, to the terminal device, the two-dimensional code with the planned path for reception added; or
a submodule for marking the planned path for reception, configured to receive a hospital plan display view returned by the hospital server, and mark the planned path for reception in the hospital plan display view, wherein the hospital plan display view is obtained after the hospital server searches out that the hospital is provided with the in-hospital navigation system according to the second query request; and
a submodule for sending the hospital plan display view, configured to send, to the terminal device, the hospital plan display view with the planned path for reception marked.

Alternatively, the business server 500 also includes:
a recommended reception time generating module, configured to generate recommended reception time according to the time of receiving the materials having not been received and target time corresponding to the target patient, wherein the target time comprises treatment time and/or hospital discharge time; and
a recommended reception time sending module, configured to send the recommended reception time to the terminal device.

Alternatively, the business server 500 also includes:
a claim request receiving module, configured to receive a claim request sent by the terminal device; and
an electronic document sending module, configured to send the electronic document of the target claim materials to the insurance server according to the claim request to make a claim application.

Alternatively, the business server 500 also includes:
a wrong information receiving module, configured to receive wrong information returned by the insurance server, wherein the wrong information is generated after the insurance server fails to search out the insurance product information purchased by the target patient according to the identification information;
a module for determining a list of preset claim materials, configured to determine a list of preset claim materials prestored in the business server;
a module for obtaining the list of preset claim materials, configured to obtain, from the hospital server, information on preset claim materials corresponding to the list of preset claim materials, wherein the information on preset claim materials comprises an electronic document of the preset claim materials and/or information for receiving the preset claim materials; and
a module for sending the information on preset claim materials, configured to send the information on preset claim materials to the terminal device.

Alternatively, the business server 500 also includes:
an item information sending module, configured to send item information not meeting a claim condition to the terminal device when the comparison result meets the claim prohibition condition, wherein the item information comprises a name of the item not meeting the claim condition and a cause for not meeting the claim condition.

In this embodiment of the present disclosure, the diagnosis and treatment information of the target patient is compared with the insurance product information purchased by the target patient to determine whether the comparison result meets the claim prohibition condition. The list of target claim materials is determined based on the insurance product information and the list of designated claim materials prestored in the business server when the comparison result does not meet the claim prohibition condition, and the target claim material information corresponding to the list of target claim materials is sent to the terminal device, such that the target patient may view, on the terminal device, the electronic document of the target claim materials and/or the information for receiving the target claim materials. In this way, the target patient can timely know types of materials required for insurance claim, and does not need to spend a lot of time to consult relevant personnel, such that time can be saved for the target patient, and experience can be improved for the target patient.

Correspondingly, an embodiment of the present disclosure further provides a business server, which comprises a processor, a memory, and a computer program stored in the memory and executed by the processor. The computer program is executable by the processor, whereby steps of the method for obtaining insurance claim materials are performed.

An embodiment of the present disclosure further provides a computer-readable medium, storing a computer program. The computer program is executable by a processor, whereby steps of the method for obtaining insurance claim materials are performed.

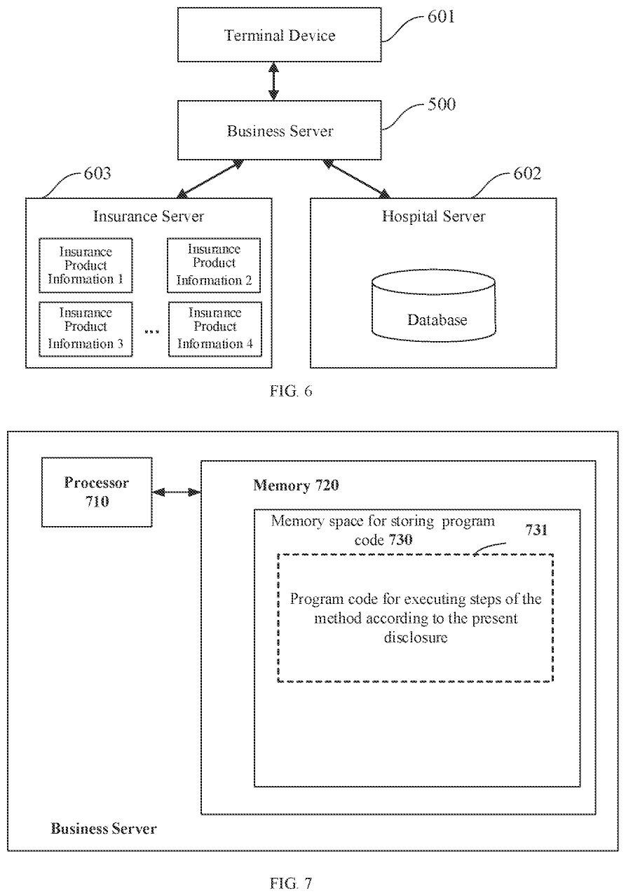

Referring to FIG. 6, FIG. 6 schematically illustrates a schematic diagram of a system for obtaining insurance claim materials according to an embodiment of the present disclosure.

An embodiment of the present disclosure further provides a system for obtaining insurance claim materials, comprising a terminal device 601, a hospital server 602, an insurance server 603, and the business server 500.

The terminal device 601 is configured to receive target claim material information corresponding to the list of target claim materials sent by the business server, a planned path for reception, and recommended reception time when the comparison result does not meet the claim prohibition condition.

The terminal device 601 is further configured to receive item information not meeting a claim condition sent by the business server when the comparison result meets the claim prohibition condition, to receive information on preset claim materials sent by the business server when the insurance server fails to search out the insurance product information purchased by the target patient, and to send a claim request to the business server.

The hospital server 602 is configured to: send medical information of the target patient to the business server; send target claim material information corresponding to the list of target claim materials to the business server, or send information on preset claim materials corresponding to the list of preset claim materials to the business server; and receive a second query request sent by the business server, and return, to the business server according to the second query request, a two-dimensional code corresponding to an in-hospital navigation system or a hospital plan display view.

The insurance server 603 is configured to: receive a first query request sent by the business server, and return, to the business server according to the first query request, the insurance product information purchased by the target patient or wrong information; and receive an electronic document of the target claim materials sent by the business server.

In this embodiment of the present disclosure, a database is deployed in the hospital server 602, and medical information of each patient is stored in the database. The business server 500 sends a request for obtaining medical information to the hospital server 602 at regular time, such that the hospital server 602 searches for medical information of the target patient according to the request for obtaining medical information, and sends the medical information of the target patient to the business server 500.

Correspondingly, the insurance server 603 also stores various insurance product information, such as insurance product information 1, insurance product information 2 to insurance product information n. When the business server 500 detects that the medical information of the target patient contains information of the preset trigger condition, the business server 500 sends a first query request to the insurance server 603, such that the insurance server 603 receives the first query request sent by the business server 500, and queries, according to the first query request, whether the insurance server stores the insurance product information purchased by the target patient. When the insurance server 603 searches out the insurance product information purchased by the target patient, the insurance server 603 returns the insurance product information purchased by the target patient to the business server 500. When the insurance server 603 does not search out the insurance product information purchased by the target patient, the insurance server 603 returns wrong information to the business server 500.

In addition, the business server 500 may extract diagnosis and treatment information from the medical information of the target patient. When the business server 500 receives the insurance product information purchased by the target patient, the business server 500 compares the diagnosis and treatment information of the target patient with the insurance product information to determine whether a comparison result meets a claim prohibition condition.

When the comparison result does not meet the claim prohibition condition, the business server 500 determines a list of target claim materials based on the insurance product information and the list of designated claim materials prestored in the business server. Next, the business server 500 sends a first acquisition request to the hospital server 602, such that the hospital server 602 queries, according to the first acquisition request, target claim material information corresponding to the list of target claim materials, and sends the target claim material information corresponding to the list of target claim materials to the business server 500. Next, the business server 500 sends the target claim material information to the terminal device 601, such that the terminal device 601 receives the target claim material information sent by the business server 500.

In addition, the business server 500 may also generate a planned path for reception according to a target location of the target patient and the site of receiving the materials having not been received. Next, the business server 500 sends a second query request to the hospital server 602. The hospital server 602 receives the second query request sent by the business server 500, and queries, according to the second query request, whether a hospital corresponding to the hospital server has an in-hospital navigation system. If the hospital has the in-hospital navigation system, the hospital server 602 returns, to the business server 500, the two-dimensional code corresponding to the in-hospital navigation system. If the hospital does not have the in-hospital navigation system, the hospital server 602 returns the hospital plan display view to the business server 500. The business server 500 adds the planned path for reception into the two-dimensional code, or marks the planned path for reception in the hospital plan display view, and sends, to the terminal device 601, the two-dimensional code with the planned path for reception added or the hospital plan display view with the planned path for reception marked, such that the terminal device 601 receives the two-dimensional code with the planned path for reception added or the hospital plan display view with the planned path for reception marked. Furthermore, the business server 500 may also generate recommended reception time according to the time of receiving the materials having not been received and target time corresponding to the target patient, and sends the recommended reception time to the terminal device 601, such that the terminal device 601 receives the recommended reception time sent by the business server 500.

When the comparison result meets the claim prohibition condition, the business server 500 sends item information not meeting a claim condition to the terminal device 601, such that the terminal device 601 receives the item information not meeting the claim condition sent by the business server 500.

However, when receiving the wrong information returned by the insurance server 603, the business server 500 determines information on preset claim materials prestored in the business server 500. The business server 500 sends a second acquisition request to the hospital server 602, such that the hospital server 602 queries, according to the second acquisition request, the information on preset claim materials corresponding to the list of preset claim materials, and sends the information on preset claim materials to the business server 500. Next, the business server 500 sends the information on preset claim materials to the terminal device 601, such that the terminal device 601 receives the information on preset claim materials sent by the business server 500.

The target patient may also make a claim application by means of the terminal device 601 and the business server 500. First, the terminal device 601 sends a claim request to the business server 500, such that the business server 500 sends an electronic document of the target claim materials to the insurance server 603 based on the claim request. Next, the insurance server 603 receives the electronic document of the target claim materials sent by the business server 500 to review the insurance claim.

In this embodiment of the present disclosure, the diagnosis and treatment information of the target patient is compared with the insurance product information purchased by the target patient to determine whether the comparison result meets the claim prohibition condition. The list of target claim materials is determined based on the insurance product information and the list of designated claim materials prestored in the business server when the comparison result does not meet the claim prohibition condition, and the target claim material information corresponding to the list of target claim materials is sent to the terminal device, such that the target patient may view, on the terminal device, the electronic document of the target claim materials and/or the information for receiving the target claim materials. In this way, the target patient can timely know types of materials required for insurance claim, and does not need to spend a lot of time to consult relevant personnel, such that time can be saved for the target patient, and experience can be improved for the target patient.

Apparatus embodiments are basically similar to method embodiments, so description of the apparatus embodiments is relatively simple, and the method embodiments can serve as reference.

The apparatus embodiments set forth above are merely exemplary, wherein units described as detached parts may be or not be detachable physically; parts displayed as units may be or not be physical units, i.e., either located at the same place, or distributed on a plurality of network units. Modules may be selected in part or in whole according to actual needs to achieve objectives of the solution of this embodiment. Those of ordinary skill in the art may comprehend and implement the embodiment without contributing creative effort.

Each of device embodiments of the present disclosure can be implemented by hardware, or implemented by software modules operating on one or more processors, or implemented by the combination thereof. A person skilled in the art should understand that, in practice, a microprocessor or a digital signal processor (DSP) may be employed to realize some or all of the functions of some or all of the parts in a computing device according to some embodiments of the present disclosure. The present disclosure may further be implemented as device or apparatus program (for example, computer program and computer program product) for executing some or all of the methods as described herein. Such program for implementing the present disclosure may be stored in a computer readable medium, or have a form of one or more signals. Such a signal may be downloaded from the Internet websites, or be provided on a carrier signal, or provided in any other form.

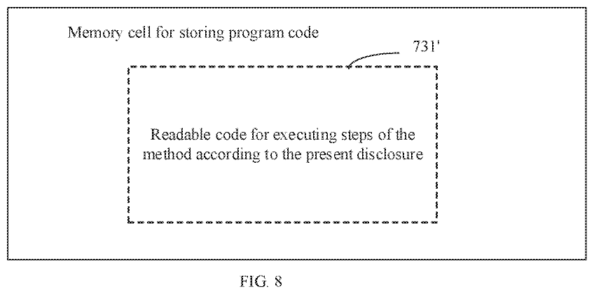

For example, FIG. 7 illustrates a business server that can implement the method according to the present disclosure. Traditionally, the business server includes a processor 710 and a computer program product or a computer-readable medium in form of a memory 720. The memory 720 may be electronic memories such as flash memory, EEPROM (Electrically Erasable Programmable Read-Only Memory), EPROM, hard disk or ROM. The memory 720 has a memory space 730 for storing program codes 731 for executing any steps in the above method. For example, the memory space 730 for storing program codes may comprise respective program codes 731 for executing the respective steps in the method as mentioned above. These program codes may be read from and/or be written into one or more computer program products. These computer program products include program code carriers such as hard disk, compact disk (CD), memory card or floppy disk. These computer program products generally are portable or stable memory cells as shown in reference FIG. 8. The memory cells may be provided with memory sections, memory spaces, etc., similar to the memory 720 of the business server as shown in FIG. 7. The program codes may be compressed for example in an appropriate form. Usually, the memory cell includes computer readable codes 731′ which can be read for example by processors 710. When these codes are operated on the business server, the business server may be caused to execute respective steps in the method as described above.

“One embodiment”, “embodiments” or “one or more embodiments” herein means that particular features, structures or characteristics described in combination with the embodiments are included in at least one embodiment of the present disclosure. Furthermore, it is to be noted that the term “in one embodiment” herein does not necessarily refer to the same embodiment.

Many details are discussed in the specification provided herein. However, it should be understood that the embodiments of the present disclosure can be practiced without these specific details. In some examples, the well-known methods, structures and technologies are not shown in detail so as to avoid an unclear understanding of the description.

In the claims, no reference mark between round brackets shall impose restriction on the claims. The word “comprise” does not exclude a component or step not listed in the claims. The wording “a” or “one” in front of a component does not exclude the presence of a plurality of such components. The present disclosure may be realized by way of hardware comprising a number of different components and by way of a suitably programmed computer. In the unit claim listing a plurality of apparatuses, some of these apparatuses may be embodied in the same hardware. The wordings “first”, “second”, and “third”, etc. do not denote any order. These wordings can be construed as naming.

Finally, it should be noted that the foregoing embodiments are merely intended for describing the technical solutions of the present disclosure, but not for limiting the present disclosure. Although the present disclosure is described in detail with reference to the foregoing embodiments, persons of ordinary skill in the art should understand that they may still make modifications to the technical solutions described in the foregoing embodiments or make equivalent replacements to some technical features thereof, which does not make corresponding technical solutions in essence depart from the spirit and the scope of the technical solutions of the embodiments of the present disclosure.

1. A method for obtaining insurance claim materials, applied to a business server, the method comprising:
sending the first query request to an insurance server; wherein the first query request comprising identification information of a target patient;
receiving insurance product information purchased by the target patient returned by the insurance server; wherein the insurance product information being obtained by querying by the insurance server according to the identification information;
comparing diagnosis and treatment information of the target patient with the insurance product information to determine whether a comparison result meets a claim prohibition condition;
determining a list of target claim materials based on the insurance product information and a list of designated claim materials prestored in the business server when the comparison result does not meet the claim prohibition condition;
obtaining, from a hospital server, target claim material information corresponding to the list of target claim materials, and sending the target claim material information to a terminal device; and
the target claim material information comprising an electronic document of the target claim materials and/or information for receiving the target claim materials.

2. The method according to claim 1, wherein a field set file is stored in the business server, and the field set file comprises:
at least one insurance type field, and a combination of insurance fields corresponding to each of the insurance type fields;
the combination of insurance fields comprising a set of insurance liability fields, a set of liability exemption fields, and a set of claim material fields;
wherein the set of insurance liability fields comprises a subset of insurance time fields, a subset of insurance content fields, and a subset of compensation manner fields, the subset of insurance time fields comprises at least one insurance time field, the subset of insurance content fields comprises at least one insurance content field, and the subset of compensation manner fields comprises at least one compensation manner field; and
the set of liability exemption fields comprises at least one liability exemption field, and the set of claim material fields comprises at least one claim material field.

3. The method according to claim 2, wherein the comparing diagnosis and treatment information of the target patient with the insurance product information to determine whether a comparison result meets a claim prohibition condition comprises:
extracting, according to an insurance type in the insurance product information, a combination of target insurance fields corresponding to a target insurance type field matching the insurance type;
the combination of target insurance fields comprising a set of target insurance liability fields and a set of target liability exemption fields;
extracting keywords comprised in each of the plurality of text messages in the insurance product information;
comparing each one of the keywords in the insurance product information with each field comprised in the set of target insurance liability fields and each field comprised in the set of target liability exemption fields respectively, to determine a first keyword matching any field in the set of target insurance liability fields and a second keyword matching any field in the set of target liability exemption fields;
extracting insurance data corresponding to the first keyword from the insurance product information;
generating first information to be compared according to a first target field in the set of target insurance liability fields matching the first keyword and the insurance data, and determining a second target field in the set of target liability exemption fields matching the second keyword as second information to be compared; and
comparing the diagnosis and treatment information of the target patient with the first information to be compared and the second information to be compared respectively, to determine whether the comparison result meets the claim prohibition condition.

4. The method according to claim 3, wherein the step of comparing the diagnosis and treatment information of the target patient with the first information to be compared and the second information to be compared respectively, to determine whether the comparison result meets the claim prohibition condition comprises:
comparing information belonging to a same category in the first information to be compared and the diagnosis and treatment information one by one, to determine whether there exists information matching the second information to be compared in the diagnosis and treatment information;
determining that the comparison result does not meet the claim prohibition condition when the information belonging to the same category matches and there does not exist the information matching the second information to be compared in the diagnosis and treatment information; and
determining that the comparison result meets the claim prohibition condition when the information belonging to the same category does not match and there exists the information matching the second information to be compared in the diagnosis and treatment information.

5. The method according to claim 3, wherein the combination of target insurance fields comprises:
a set of target claim material fields;
the step of determining a list of target claim materials based on the insurance product information and a list of designated claim materials prestored in the business server when the comparison result does not meet the claim prohibition condition comprises:
comparing each of the keywords in the insurance product information with each field comprised in the set of target claim material fields respectively when the comparison result does not meet the claim prohibition condition, to determine a third keyword matching any field in the set of target claim material fields; and
determining a third target field in the set of target claim material fields matching the third keyword as the list of target claim materials.

6. The method according to claim 2, wherein before the step of sending the first query request to an insurance server, the method further comprises:
obtaining medical information of the target patient from the hospital server at regular time; and
performing the step of sending the first query request to an insurance server when the medical information comprises information meeting a preset trigger condition.

7. The method according to claim 6, wherein before the step of comparing diagnosis and treatment information of the target patient with the insurance product information to determine whether a comparison result meets a claim prohibition condition, the method further comprises:
determining information in the medical information belonging to a same category as a category of any field comprised in the field set file as the diagnosis and treatment information of the target patient.

8. The method according to claim 1, wherein the diagnosis and treatment information of the target patient comprises:
a list of materials having been received;
the step of obtaining, from a hospital server, target claim material information corresponding to the list of target claim materials, and sending the target claim material information to a terminal device comprises:
determining a list of materials not matching the list of materials having been received from the list of target claim materials as a list of materials having not been received;
obtaining, from the hospital server, an electronic document of materials having been received corresponding to the list of materials having been received, an electronic document of materials having not been received corresponding to the list of materials having not been received, and information for receiving the materials having not been received; and
sending, to the terminal device, the electronic document of materials having been received and/or information for receiving the materials having been received, and the electronic document of materials having not been received and/or the information for receiving the materials having not been received;
wherein the information for receiving the materials having been received comprises: a material name of the materials having been received and first marking information of the materials having been received, the first marking information being configured for indicating that the materials having been received have been received; the information for receiving the materials having not been received comprises a material name of the materials having not been received, second marking information of the materials having not been received, and time, site and attentions of receiving the materials having not been received, the second marking information being configured for indicating that the materials having not been received have not been received.

9. The method according to claim 8, wherein after the step of obtaining, from a hospital server, target claim material information corresponding to the list of target claim materials, and sending the target claim material information to a terminal device, the method further comprises:
generating a planned path for reception according to a target location of the target patient and the site of receiving the materials having not been received; and
sending the planned path for reception to the terminal device.

10. The method according to claim 9, wherein the step of sending the planned path for reception to the terminal device comprises:
sending a second query request to the hospital server;
receiving a two-dimensional code corresponding to an in-hospital navigation system returned by the hospital server, and adding the planned path for reception into the two-dimensional code;
the two-dimensional code being obtained after the hospital server searches out that a hospital is provided with the in-hospital navigation system according to the second query request;
sending, to the terminal device, the two-dimensional code with the planned path for reception added; or
receiving a hospital plan display view returned by the hospital server, and marking the planned path for reception in the hospital plan display view;
the hospital plan display view being obtained after the hospital server searches out that the hospital is provided with the in-hospital navigation system according to the second query request; and
sending, to the terminal device, the hospital plan display view with the planned path for reception marked.

11. The method according to claim 8, wherein after the step of obtaining, from a hospital server, target claim material information corresponding to the list of target claim materials, and sending the target claim material information to a terminal device, the method further comprises:
generating recommended reception time according to the time of receiving the materials having not been received and target time corresponding to the target patient;
the target time comprising treatment time and/or hospital discharge time; and
sending the recommended reception time to the terminal device.

12. The method according to claim 1, wherein after the step of obtaining, from a hospital server, target claim material information corresponding to the list of target claim materials, and sending the target claim material information to a terminal device, the method further comprises:
receiving a claim request sent by the terminal device; and
sending the electronic document of the target claim materials to the insurance server according to the claim request to make a claim application.

13. The method according to claim 1, wherein after the step of sending the first query request to an insurance server, the method further comprises:
receiving wrong information returned by the insurance server;
the wrong information being generated after the insurance server fails to search out the insurance product information purchased by the target patient according to the identification information;
determining a list of preset claim materials prestored in the business server;
obtaining, from the hospital server, information on preset claim materials corresponding to the list of preset claim materials, the information on preset claim materials comprising an electronic document of the preset claim materials and/or information for receiving the preset claim materials; and
sending the information on preset claim materials to the terminal device.

14. The method according to claim 1, wherein after the step of comparing diagnosis and treatment information of the target patient with the insurance product information to determine whether a comparison result meets a claim prohibition condition, the method further comprises:
sending item information not meeting a claim condition to the terminal device when the comparison result meets the claim prohibition condition; and
the item information comprising a name of the item not meeting the claim condition and a cause for not meeting the claim condition.

15. (canceled)

16. A business server, comprising a processor, a memory, and a computer program stored in the memory and executed by the processor, wherein the computer program is executable by the processor, whereby steps of the method for obtaining insurance claim materials according to claim 1 is performed.

17. (canceled)

18. A non-volatile computer-readable storage medium, storing a computer program, wherein the computer program is executable by a processor, whereby steps of the method for obtaining insurance claim materials according to claim 1 is performed.

19. A system for obtaining insurance claim materials, comprising a terminal device, a hospital server, an insurance server, and a business server according to claim 16;
wherein the terminal device is configured to receive target claim material information corresponding to the list of target claim materials sent by the business server, a planned path for reception, and recommended reception time when the comparison result does not meet the claim prohibition condition;
wherein the terminal device is further configured to receive item information not meeting a claim condition sent by the business server when the comparison result meets the claim prohibition condition, and to receive information on preset claim materials sent by the business server when the insurance server fails to search out the insurance product information purchased by the target patient;
wherein the terminal device is further configured to send a claim request to the business server;
wherein the hospital server is configured to: send medical information of the target patient to the business server; send target claim material information corresponding to the list of target claim materials to the business server, or send information on preset claim materials corresponding to the list of preset claim materials to the business server; and receive a second query request sent by the business server, and return, to the business server according to the second query request, a two-dimensional code corresponding to an in-hospital navigation system or a hospital plan display view; and
wherein the insurance server is configured to: receive a first query request sent by the business server, and return, to the business server according to the first query request, the insurance product information purchased by the target patient or wrong information; and
receive an electronic document of the target claim materials sent by the business server.

20. The method according to claim 13, wherein the business server receives the wrong information returned by the insurance server, the method further comprises:
determining that the target patient has not purchased the insurance, and the business server sends a second reminder message to the terminal device to remind the target patient that no insurance purchase record is searched out, and remind the target patient whether to obtain the information on preset claim materials.

21. The method according to claim 13, wherein the terminal device receives a second acknowledged operation from the target patient, the method further comprises:
the terminal device sends a request for obtaining the information on preset claim materials to the business server, the business server determines a list of preset claim materials prestored in the business server according to the request for obtaining the information on preset claim materials.

22. The method according to claim 21, wherein the list of preset claim materials is determined, the method further comprises:
the business server sends a second acquisition request to the hospital server, the second acquisition request includes the list of preset claim materials prestored in the business server, the hospital server queries, based on the second acquisition request, the information on preset claim materials corresponding to the list of preset claim materials prestored in the hospital server, and sends the information on preset claim materials to the business server, the information on preset claim materials includes the electronic document of the preset claim materials and/or the information for receiving the preset claim materials.
